(19) United States
(12) Patent Application Publication
(10) Pub. No.: US 2008/0234628 A1
(43) Pub. Date: Sep. 25, 2008

(54) MULTIPLE PART ELECTROTRANSPORT DRUG DELIVERY DEVICE

(76) Inventors: Wanda Dent, Chanhassen, MN (US); Johannes N. Gaston, Minnetonka, MN (US); Douglas J. Vanornum, Menasha, WI (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/052,575

(22) Filed: Mar. 20, 2008

Related U.S. Application Data
(60) Provisional application No. 60/896,398, filed on Mar. 22, 2007.

(51) Int. Cl. A61N 1/30; B23P 11/00

(52) U.S. Cl. 604/20; 29/428

(57) ABSTRACT

An electrotransport device for delivering a therapeutic agent through a body surface of a patient. The device has an electronic module that can be coupled with a reservoir module to form the electrotransport device. The reservoir module has a reservoir that contains the therapeutic agent for delivery through the body surface by electrotransport. The reservoir module also has a cutout forming a channel having a narrow channel portion and a less narrow channel portion with channel walls. The electronic module has circuitry for electrically driving the therapeutic agent for electrotransport and having a body with a narrow portion and a less narrow portion corresponding to the narrow channel portion and the less narrow channel portion. The narrow portion of the electronic module can be guided into the narrow channel portion through the less narrow channel portion of the reservoir module.

### CROSS REFERENCE TO RELATED U.S. APPLICATION DATA

The present application is derived from and claims priority to provisional applications U.S. Ser. No. 60/896,398, filed Mar. 22, 2007, which is herein incorporated by reference in its entirety.

### TECHNICAL FIELD

The present invention relates to a multiple part electrotransport drug delivery system for driving ionic drug across a body surface or membrane. In particular, the invention relates to a system having an electronic part and a drug reservoir part that can be coupled together before drug delivery.

### BACKGROUND

The delivery of active pharmaceutical agents through the skin provides many advantages, including comfort, convenience, and non-invasiveness. Gastrointestinal irritation and the variable rates of absorption and metabolism including first pass effect encountered in oral delivery are avoided. Transdermal delivery also provides a high degree of control over blood concentrations of any particular active agent.

The natural barrier function of the body surface, such as skin, presents a challenge to delivery therapeutics into circulation. Devices have been invented to provide transdermal delivery of drugs. Transdermal drug delivery can generally be considered to belong to one of two groups: transport by a “passive” mechanism or by an “active” transport mechanism. In the former, such as fentanyl transdermal systems available from Jassen Pharmaceuticals and other drug delivery skin patches, the drug is incorporated in a solid matrix, a reservoir with rate-controlling membrane, and/or an adhesive system.

Passive transdermal drug delivery offers many advantages, such as ease of use, little or no pain at use, disposability, good control of drug delivery and avoidance of hepatic first-pass metabolism. However, many active agents are not suitable for passive transdermal delivery because of their size, ionic charge characteristics, and hydrophilicity. Most passive transdermal delivery systems are not capable of delivering drugs under a specific profile, such as by ‘on-off’ mode, pulsatile mode, etc. Consequently, a number of alternatives have been proposed where the flux of the drug(s) is driven by various forms of energy. Some examples include the use of iontophoresis, ultrasound, electroporation, heat and microneedles. These are considered to be “active” delivery systems.

One method for transdermal delivery of such active agents involves the use of electrical current to actively transport the active agent into the body through intact skin by electrotransport. Electrotransport techniques may include iontophoresis, electroosmosis, and electroporation. Electrotransport devices, such as iontophoretic devices are known in the art, see, e.g., U.S. Pat. Nos. 5,057,072, 5,084,008, 5,147,297, 6,039,977, 6,049,733, 6,171,294, 6,181,963, 6,216,033, and US Patent Publication 20030191946. One electrode, called the active or donor electrode, is the electrode from which the active agent is delivered into the body. The other electrode, called the counter or return electrode, serves to close the electrical circuit through the body. In conjunction with the patient's body tissue, e.g., skin, the circuit is completed by connection of the electrodes to a source of electrical energy, and usually to circuitry capable of controlling the current passing through the device. If the substance to be driven into the body is ionic and is positively charged, then the positive electrode (the anode) will be the active electrode and the negative electrode (the cathode) will serve as the counter electrode. If the ionic substance to be delivered is negatively charged, then the cathodic electrode will be the active electrode and the anodic electrode will be the counter electrode.

A prior iontophoretic system similar to that of U.S. Pat. No. 6,181,963 is shown in FIG. 1. FIG. 1 shows a perspective exploded view of an electrotransport device 10 having an activation switch in the form of a push button switch 12 and a display in the form of a light emitting diode (LED) 14. Device 10 includes an upper housing 16, a circuit board assembly 18, a lower housing 20, anodic electrode 22, cathodic electrode 24, anodic reservoir 26, cathodic reservoir 28 and skin-compatible adhesive 30. Upper housing 16 has lateral wings 15 that assist in holding device 10 on a patient's skin. Upper housing 16 is preferably composed of an injection moldable polymer.

Printed circuit board assembly 18 includes an integrated circuit 19 coupled to discrete electrical components 40 and battery 32. Printed circuit board assembly 18 is attached to housing 16 by posts (not shown) passing through openings 13a and 13b, the ends of the posts being heated/melted in order to heat weld the circuit board assembly 18 to the housing 16. Lower housing 20 is attached to the upper housing 16 by means of adhesive 30, the upper surface 34 of adhesive 30 being adhered to both lower housing 20 and upper housing 16 including the bottom surfaces of wings 15.

Shown (partially) on the underside of printed circuit board assembly 18 is a battery 32, preferably a button cell battery and most preferably a lithium cell. Other types of batteries may also be employed to power device 10.

The circuit outputs (not shown in FIG. 1) of the circuit board assembly 18 make electrical contact with the electrodes 24 and 22 through openings 23,23′ in the depressions 25, 25′ formed in lower housing, by means of electrically conductive adhesive strips 42, 42′. Electrodes 22 and 24, in turn, are in direct mechanical and electrical contact with the top sides 44′, 44 of reservoirs 26 and 28. The bottom sides 46′, 46 of reservoirs 26,28 contact the patient's skin through the openings 29′, 29 in adhesive 30. The skin-facing side 36 of the adhesive 30 has adequate adhesive property to maintain the device on the skin for the duration of the use of the device.

Recently, there have been suggestions to provide different parts of an electrotransport system separately and connect them together for use. For example, such connected-together systems might provide advantages for reusable controller circuit. In reusable systems, the drug-containing units are disconnected from the controller when the drug becomes depleted and a fresh drug-containing unit is then connected to the controller again. Examples of electrotransport devices having parts being connected together before use include those described in U.S. Pat. No. 5,320,597 (Sage, Jr. et al); U.S. Pat. No. 4,731,926 (Sibalis), U.S. Pat. No. 5,358,483 (Sibalis), U.S. Pat. No. 5,135,479 (Sibalis et al.), UK Patent Publication GB2239803 (Devane et al), U.S. Pat. No. 5,919,155 (Lattin et al.), U.S. Pat. No. 5,445,609 (Lattin et al.), U.S. Pat. No. 5,603,693 (Frenkel et al.), and WO1996036394 (Lattin et al.).

However, many of the prior connected-together systems are cumbersome to use and do not provide for easy assembly and use.

What is needed is an electrotransport device in which the electronic part and the reservoir part can be easily assembled about the time of use.

### SUMMARY

The present invention relates to an electrotransport device for delivering a therapeutic agent through a body surface of a patient. The device has an electronic module that can be coupled with an agent module (e.g., reservoir module) before use to provide circuitry for driving the drug, thereby forming the assembled electrotransport device. The present invention provides such electrotransport devices and methods of making and using such electrotransport devices.

In one aspect, the agent module has a compartment (e.g., reservoir) containing the therapeutic agent for delivery through the body surface by electrotransport. The agent module also has a cutout forming a channel having a narrow channel portion and a less narrow channel portion. The channel has channel walls. The electronic module has circuitry for electrically driving the therapeutic agent for electrotransport and has a body with a narrow portion and a less narrow portion corresponding to the narrow portion and the less narrow portion of the channel. The narrow portion of the electronic module can be guided into the narrow channel portion through the less narrow channel portion of the agent module.

Because of the shapes of the electronic module and the agent module of the present invention, the two modules can easily be aligned to fit together in correct orientation and therefore correct electrical polarity. Further, due to the presence of wide (or less narrow) and narrow portions of the channel in the agent module, the electronic module can be guided into position to fit with the agent module. Also, because of the corresponding shapes of the electronic module and the agent module, the two modules can be match fitted together to result in tight seams (junctions) to provide protection against liquid and other unintended intrusion of unwanted material.

In an aspect, the present invention provides a method of making an electrotransport device for delivering a therapeutic agent through a body surface of a patient. The method includes coupling an agent module to an electronic module, wherein the agent module has a cutout forming a channel having a narrow channel portion and a less narrow channel portion with channel walls. The electronic module has circuitry for electrically driving the therapeutic agent for electrotransport. The electronic module further has a body with a narrow portion and a less narrow portion corresponding to the narrow portion and the less narrow portion of the channel. The method includes guiding the narrow portion of the electronic module into the narrow channel portion through the less narrow channel portion of the agent module to couple the modules together.

In another aspect, an electrotransport device is provided in which the device has an agent module that can be coupled with an electronic module, which is multilayered. In one aspect, the electronic module has an upper cover and a lower cover sandwiching or surrounding about and protecting a printed circuit board, which contains circuitry for electrically driving the therapeutic agent for electrotransport. The agent module has a compartment (e.g., reservoir) containing the therapeutic agent for delivery through the body surface by electrotransport. In one aspect, the agent module has a cutout forming a channel having a narrow channel portion and a less narrow channel portion with channel walls; and the electronic module has a body with a narrow portion and a less narrow portion corresponding to the narrow portion and the less narrow portion of the channel. The narrow portion of the electronic module can be guided into the narrow channel portion through the less narrow channel portion of the agent module. Having multiple layers, the electronic module can provide a resilient top layer for tight seal with the agent module and yet provide stiff structures to protect the printed circuit board and to firmly couple with corresponding stiff structures in the agent module. Thus, the present invention provides advantageous devices that are sturdy and yet well protected against liquid penetration at externally visible seams (where the electronic module and the agent module surfaces meet). The multilayered construction of the electronic module, and preferably of the agent module in certain embodiments allows for appropriate placement of the fitting portions. Because the layers can be made separately and then affixed together, either by mechanical anchoring, chemical bonding or by molding together by heat, the parts that need to be fitted together can be positioned at strategic locations for optimal cooperative operation. The layered construction of the electronic module and the agent module provides advantages in making and positioning of the couplers. The layered construction further provides protection of the electronics from mechanical disturbance and moisture as well as protection of the reservoir(s) from mechanical disturbance.

The present invention also provides methods of making and methods of using the above electrotransport devices. After the electronic module has been coupled to the agent module, the device is applied onto the body surface of a patient.

It is to be understood that the present invention of engaging two modules can be applied to electrotransport devices such as iontophoretic devices, electroosmosis devices, and electroporation devices, as long as there are two modules that need to be coupled together for mechanical and electrical engagement.

### BRIEF DESCRIPTION OF THE FIGURES

The present invention is illustrated by way of examples in embodiments and not limitation in the figures of the accompanying drawings in which like references indicate similar elements. The figures are not shown to scale unless indicated otherwise in the content.

FIG. 1 illustrates an exploded perspective view of a prior art typical electrotransport system.

FIG. 2 illustrates an exploded perspective view of an embodiment of an electrotransport system of the present invention.

FIG. 3 illustrates a perspective view of an embodiment of an electrotransport system of the present invention being assembled.

FIG. 4 shows a schematic side view of an embodiment like FIG. 2 after being assembled.

FIG. 5 shows a top view of an embodiment similar to FIG. 2 after being assembled.

FIG. 6 shows a schematic side view of an embodiment like FIG. 2 being assembled.

FIG. 7 shows a schematic perspective view of an embodiment of an electrical connector (receptor) for an electrotransport system of the present invention.

FIG. 8 shows a schematic perspective top view of an embodiment of another electrical connector for an electrotransport system of the present invention.

FIG. 9 shows a schematic perspective bottom view of the embodiment of the electrical connector of FIG. 8.

### DETAILED DESCRIPTION

The present invention is directed to an electrotransport drug delivery system that has two parts which are assembled together before drug administration to a patient. In particular, the system includes an agent-containing module (“agent module” for short) having a compartment (e.g., reservoir) containing the drug (or therapeutic agent) and an electronic module for coupling to the agent module to drive the drug in electrotransport through a body surface.

The practice of the present invention will employ, unless otherwise indicated, conventional methods used by those skilled in the art of mechanical and electrical connections in drug device development.

In describing the present invention, the following terminology will be used in accordance with the definitions set out below.

The singular forms “a,” “an” and “the” include plural referents unless the context clearly dictates otherwise. Thus, for example, reference to “a polymer” includes a single polymer as well as a mixture of two or more different polymers.

### MODES OF CARRYING OUT THE INVENTION

The present invention provides an electrotransport device that is assembled before use for electrotransport delivery of ionic compounds (e.g., ionic drugs such as fentanyl and analogs, polypeptides, and the like) through a surface, such as skin.

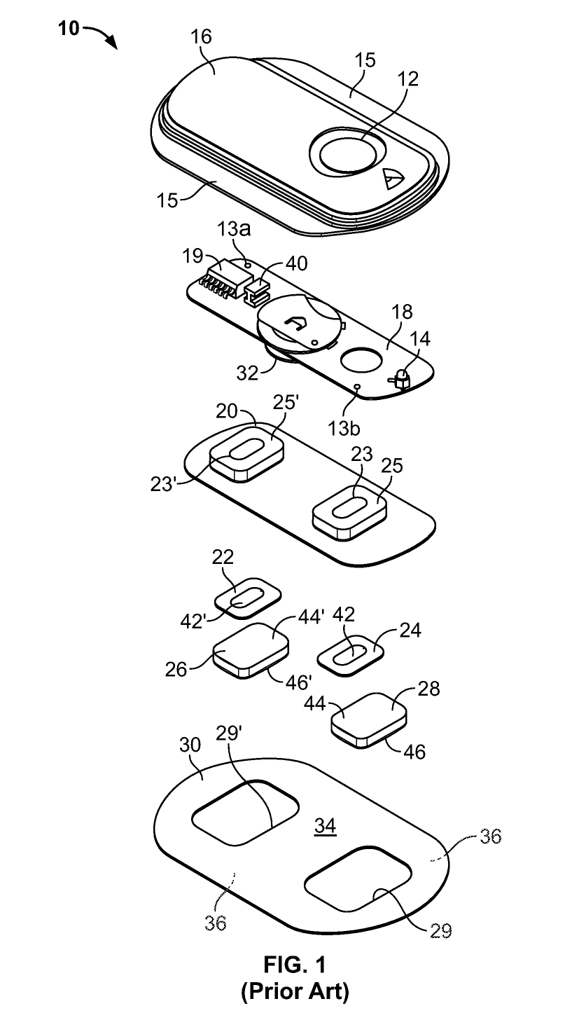

Electrotransport devices, such as iontophoretic devices are known in the art, e.g., U.S. Pat. No. 6,216,033. The structures, drugs, and electrical features of U.S. Pat. No. 6,216,033 and in FIG. 1 can be adapted to equivalents to be used in the present invention, as can be understood by one skilled in the art. In an iontophoretic drug delivery device, there is a drug reservoir and a counter reservoir.

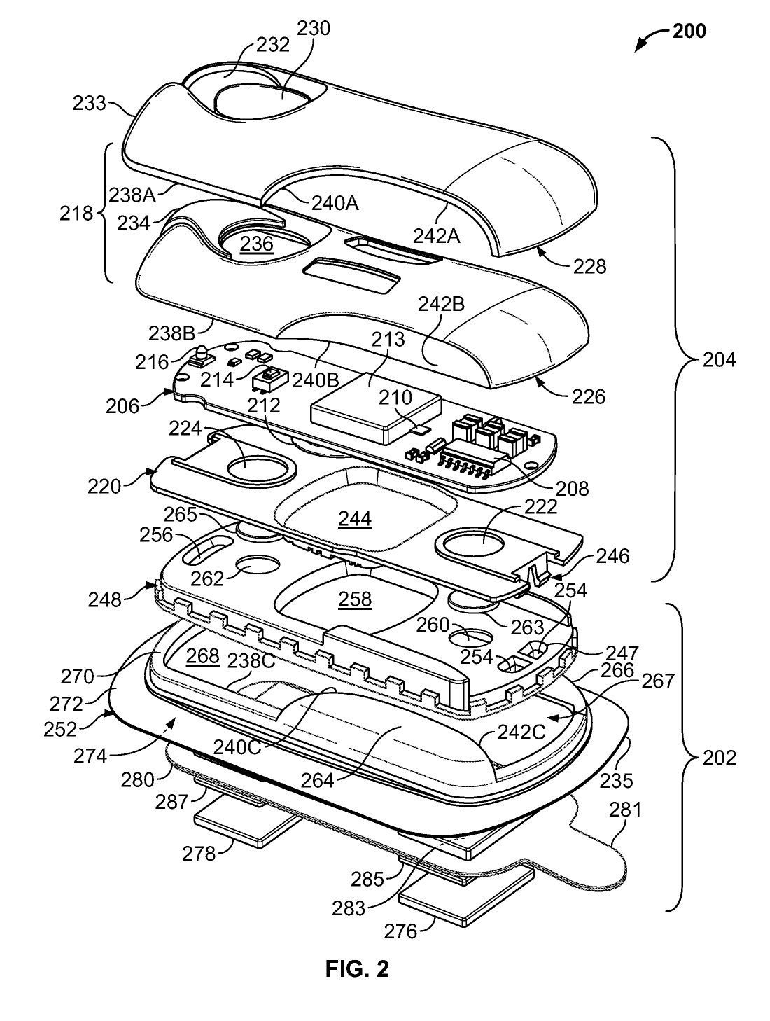

FIG. 2 shows an embodiment of an electrotransport device of the present invention. The electrotransport device 200 includes an agent-containing module (or in this embodiment, reservoir module) 202 and an electronic module 204. The electronic module 204 includes printed circuit board (PCB) assembly 206 that includes an integrated circuit 208 coupled to discrete electrical components 210 and power source (e.g., battery) 212. A switch 214 is connected to the integrated circuit 208 for together controlling the operation of the electrotransport device 200. An optical display (e.g., LED) 216 acts as an indicator for the operation of the device 200 to show functions of the device 200, e.g., indicating whether the device is in a drug delivery mode, the amount of drug that has been delivered up to the moment, the number of doses the device has been activated to deliver, etc. An alternative display would be a digital or alpha-numeric display (e.g., liquid crystal display) for showing the operation functions and parameters of the device. Information that can be displayed includes the number of doses already delivered. Examples of electronic components that can be present include audible alarm (shown as transducer 213) to alert the user of undesirable conditions, successful initiation of dose delivery, etc., and other features that improve or support the functions of the integrated circuit, display, etc.

The PCB assembly 206 is sandwiched between an upper cover (or top cover) 218 and a lower cover (or bottom cover) 220 so that the PCB assembly 206 is enclosed and protected by them except for electrical connectors (not shown in FIG. 2 because it is hidden in the perspective view) that are positioned at openings (holes) 222 and 224 of the lower cover 220 for electrically connecting to corresponding connectors in the reservoir module 202. Mechanical connection and engagement that secure or lock the connectors together can also be present.

The upper cover 218 of the electronic module 204 includes a lower layer 226 made of rigid or semirigid material (e.g., polypropylene) and an upper layer 228 made of a less rigid elastomer, such as ethylene vinyl acetate or ethylene-octene copolymer, e.g., ethylene-octene copolymers available under the tradename ENGAGEL® from Dow Chemical Company. The polymeric material from which the upper layer 228 is made is softer and more resilient than the polymeric material of the lower layer 226 so that when the electronic module is coupled to match with the reservoir module 202 the upper layer 228 can match contours snuggly with the reservoir module to provide a splash-water resistant or liquid resistant (drip-proof) seam, as well as provide visual confirmation of correct assembly. In other words, liquid will not penetrate to cause failure of the device through the liquid resistant seam during occasional momentary water exposure such as splashing as under a short spray. Further, by using materials that are hydrophobic and/or that can butt tightly, the seam can be made to keep out aqueous liquid such as water in normal daily routine use. For example, the material at the seam can have a coating of a hydrophobic material such as polytetrafluoroethylene. A button cover portion 230 of the upper layer 228 is adequately flexible and soft such that finger pressure by a finger pressing on the button cover portion 230 can activate or deactivate the switch 214. It is noted that other elastomers or semi-rigid polymers can also be used so long as it provides an adequate amount of resiliency.

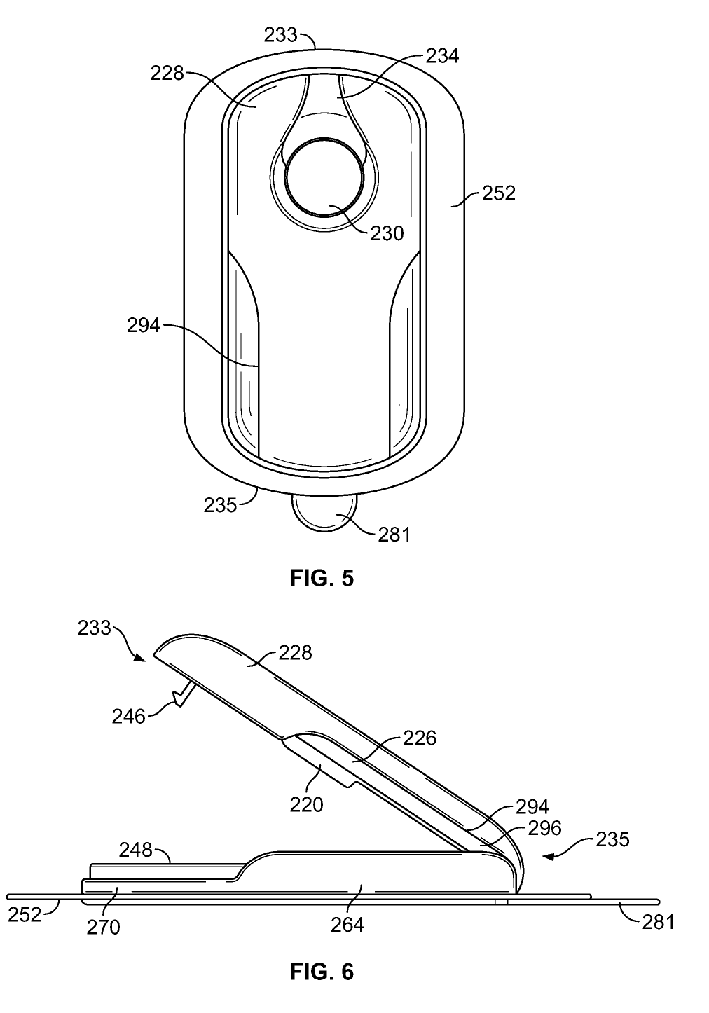

A cutout 232 on the upper layer 228 at the anterior end 233 of the device 200 allows light transmitted from the display 216 to be visible from the top view. Of course, in alternative designs, the display itself can also include LED, digital display, etc. for displaying information. For example, the embodiment in FIG. 5 has a display 234 that can be a digital display. For the sake of convenience of illustration, in this and similar embodiments, the end having the display 216 is considered the “anterior” end and the direction toward the upper layer 228 from the reservoir module 202 is considered “top” and “upper” whereas the direction towards the reservoir module 202 from the upper layer 228 is considered “bottom” or “lower” herein. The end opposite to the anterior end 233 is considered the posterior end 235 and a line traversing from the anterior end to the posterior end is considered to traverse longitudinally.

In this embodiment, the lower layer 226 of the upper cover 218 of the electronic module 204 is made of a transparent or translucent polymeric material that is stiffer than the upper layer 228 to protect the PCB assembly 206. A window portion 234 fits into the cutout 232 in the upper layer 228 and allows light emitted from the display 216 to be seen through the transparent or translucent window portion 234 from a top view. A cutout (opening) 236 on the lower layer 226 above the display 216 allows the display 216 to be viewed through the transparent or translucent window portion 234. The cutout 236 can alternatively be covered by a transparent or translucent material for the display 216 to be seen. Useful transparent or translucent polymeric material for the window 234 includes acrylic, polycarbonate, polyethylene, polypropylene, polyethylene terephthalate, and the like. Further, glass fibers, glass particles, silica, and the like can also be included in the transparent or translucent polymeric material to provide more stiffness, to provide support and protection of the PCB assembly 206 and to secure to the lower cover 220 of the electronic module 204. Additives to enhance the bonding in the polymeric materials and dispersion aids to improve dispersion of additives or light in the transparent or translucent material can also be used in the molding material. For a digital display, a window with sufficient transparency for the digits to be read is provided.

The upper layer 228 has a wide portion 238A gradually narrowing from the wide portion 238A through a narrowing portion 240A to a narrow portion 242A. The lower layer 226 also has a wide portion 238B gradually narrowing from the wide portion 238B through a narrowing portion 240B to a narrow portion 242B. The wide portion 238A, narrowing portion 240A, and narrow portion 242A of the upper layer 228 are generally the same size or slightly wider in the lateral direction than the wide portion 238B, narrowing portion 240B, and narrow portion 242B of the lower layer 226 such that the wide portion 238A, narrowing portion 240A, and narrow portion 242A of the upper layer 228 can extend to match contours with the wide portion 238C, narrowing portion 240C, and narrow portion 242C of the outer upper portion 252 of the reservoir module 202, resulting in a puzzle-piece fit. A puzzle-piece fit means a matching fit between two pieces as appears in a jig-saw puzzle.

The lower cover 220 of the electronic module 204 has a cavity 244 for accommodating the battery 212 and has openings 222 and 224 allowing electrical connectors from the PCB assembly 206 to electrically connect with the reservoir module 202. The lower cover 220 further has couplers 246, 257, which can contain one, two, or more bent lips (or angled ledges because they are ledges or lips that are bent at an angle). Coupler 257 at the anterior end is not visible from FIG. 2 because it is hidden in the perspective view. The couplers 246, 257 can lock with a receptor couplers 247, 256 respectively from the reservoir module 202, providing a snap or click fit (i.e., one piece can be inserted into another with a snap or click as a resilient portion of a piece is squeezed initially by a restriction and suddenly released from the squeeze once the portion has passed the restriction). Alternatively, the openings 222 and 224 instead of being open voids rather can be conductive pads that are electrically and mechanically connected to the electronics in the PCB. The conductive pads allow the electrical connectors from the reservoir module 202 to connect electrically to the electronic module 204.

The reservoir module 202 is typically a disposable unit that can be discarded after use with appropriate procedure. The reservoir module 202 has a rigid inner upper portion 248 and has a less rigid outer upper portion 252 surrounding the inner upper portion 248 laterally and at the anterior end 233 and the posterior end 235. The inner upper portion has a generally layer shape. At one end of the device, the inner upper portion 248 has coupler receptor 247 having one or more openings 254 for lockingly receiving the coupler 246 of the electronic module 204. At another end of the device, another receptor 256 with opening(s) is present to lockingly receive another coupler insert(s) (not shown because it is hidden in the perspective view) extending from the lower cover 220 of the electronic module 204. The couplers at the two ends 233, 235 can have different structures for securing the two modules together. The couplers at the two ends can both have a single insert and receptor hole, or two or more inserts and receptor holes, or one end can have inserts and receptor holes different from the other end. Inserts and receptor holes can be located at either the electronic module side or the reservoir module side.

A cavity 258 in the inner upper portion 248 provides for space to accommodate the portion of the lower cover protecting the battery 212. Openings 260, 262 securely accommodate electrical connectors 263, 265 that provide electrical connection between the electrical connectors of the electronic module 204 and the electrode current distributor 285, 287. The electrical connectors 263, 265 have grooves or other securing features for securing them to the inner upper portion 248 at the openings 260, 262 and ensuring good electrical connection and, optionally, mechanical securing connection. The openings 260,262 can have rims around them to fit into the grooves of the electrical connectors 263, 265 for interference fit for securing together, or for ensuring good electrical connection by digging into the material of electrical connectors 263, 265. The electrical connectors 263, 265 can be made of metal or carbonized polymer to make them conductive. Alternatively, the electrical connectors 263, 265 can be comolded with the inner upper portion 248.

The inner upper portion 248 in the reservoir module 202 and the lower cover 220 in the electronic module 204 can be made with relatively stiff material, preferably electrically insulating polymeric material so that they can be coupled together to provide a sturdy support structure for the PCB assembly 206 and the flat flange (or wing) 272 of the outer upper portion 252 in the reservoir module 202. A layer of adhesive can be provided under the flat flange (or wing) 272 for attachment to the body surface. Useful material for making the inner upper portion 248 and the lower cover 220 include polyethylene, polypropylene, polyethylene terephthalate, polystyrene, and the like. Glass fibers, glass particles, silica, and the like can also be included in the polymeric material to provide more stiffness. When two materials are molded together, they are selected so that they are compatible for comolding, e.g., having similar thermal and chemical property. Further, pigments and other material can also be included in the construction material for the pieces that provide mechanical support. The stiff material also provides a means to create secure mechanical attachment that may be independent of the electrical connection.

The outer upper portion 252 in the reservoir module 202 includes a cutout 268 for receiving and securing the more rigid inner upper portion 248. Means for securing the various portions and pieces together can include couplers such as mating insert and receptors, adhesive, frictionally interfering edges, etc. Towards the posterior end 235, the outer upper portion 252 includes an upwardly extending ridges 264, 266. The ridges 264, 266 edge a channel 267 through which the posterior portion of the electronic module 204 can be received. The ridges 264, 266 each have a narrowing portion 240C and narrow portion 242C corresponding to and for receiving the narrowing portion 240B and narrow portion 242B of the lower layer 226 of the upper cover 218 in the electronic module 204. Further, the narrowing portion 240C and narrow portion 242C of the outer upper portion 252 of the reservoir module 202 correspond to and intimately match fit the narrowing portion 240A and the narrow portion 242A of the of the upper layer 228 (of the upper cover 218) in the electronic module 204 to provide a seam that is liquid resistant. The seam also provides visual indication that the electronic module 204 and the reservoir module 202 are properly and completely assembled. The ridges 264, 266 run generally from about the posterior end 235 of the outer upper portion 252 to half way or more, preferably to about 50-75% the longitudinal length of the reservoir module 202. Because of the narrowing portion 240C and narrow portion 242C of the outer upper portion 252 the ridges 264, 266 have a width (i.e., in a direction inwardly toward each other) in the lateral direction.

The outer upper portion 252 also has a frame 270 framing the cutout 268. The ridges 264, 266 rise above the frame 270 to receive the corresponding portions of the electronic module. The frame 270 has an inner perimeter to receive the inner upper portion 248 such that when the inner upper portion 248 of the reservoir module 202 is fitted into the outer upper portion 252, the inner upper portion can accommodate the lower cover 220 of the electronic module 204. Towards the anterior end 233, where the ridges 264, 266 are absent, the wide portion 238A of the upper layer 228 in the electronic module 204 can match with the frame 270 to provide a seam that is tight to indicate properly assembly and prevent ingress of foreign material. The outer upper portion 252 in the reservoir module 202 has a thin, generally flat annular flange 272 extending from the frame 270 all around to provide a lower surface 274 under the flat annular flange 272 for adhesive attachment to a body surface (e.g., human skin) when the device 200 is applied to the patient. Adhesive is not shown in FIG. 2 for the sake of clarity of the figure. Other than an annular shape, the thin flat flange can have the form of wings on the lateral sides of the outer upper portion 252 (similar to wings 15 shown in FIG. 1). The thin flat flange 272, because of its flexibility, can conform to the contour of the skin surface. As used herein, “annular” means ring-shaped, whether it be exactly circular, off-circular, oval, oblong, or shaped like a race track in a stadium. The outer upper portion 252 is made of a relatively soft, pliable, resilient material such as an elastomer, e.g., ethylene octene copolymer, silicone, butyl rubber, etc. Preferably the material is a biocompatible polymer, e.g., ethylene octene copolymer (see US patent publication US20020128591) that does not absorb the beneficial agent or interact with the adhesive, or other material in the reservoir, such as the matrix material of the hydrogel.

In this embodiment, the reservoir module 202 has reservoirs (preferably hydrogel) 276, 278 on the under side of the reservoir module 202 for contacting body surface of a patient for electrotransport of ions. A lower layer 280 in the reservoir module 202 is located at and secured to the underside of the upper inner portion 248. The lower layer 280 has downwardly facing cavities 283 for accommodating current distributors 285, 287 and reservoirs 276, 278. If desired, a tab 281 can extend off one end (e.g., posterior end 235) of the lower layer 280 in the reservoir module 202. An authorized person (such as a medical worker, e.g., doctor or nurse) can grasp the tab 281 to pull off the lower layer 280 with the reservoirs 276, 278 from the reservoir module 202 for disposal according to controlled pharmaceutical disposal regulations after the prescribed electrotransport delivery by the device 200 is completed. In this way, the risk for drug abuse through illicit use of the device is reduced.

Although it is possible to include electronic components in the reservoir module, to reduce the complexity of the reservoir module, reduce the risk of electronics failing because of corrosion due to the presence of liquid and moisture, and result in easier manufacturing processes, it is preferred that the reservoir module contains no active electronic components such as transistor, integrated circuit, operational amplifier, etc. Active electronic components are those that can provide gain in an electrical circuit, such as transistors, field effect transistors, triodes, etc. Preferably the only electrical components present in the reservoir module are nonactive components. In some embodiments, the only electrical material present in the reservoir module is conductor leading to the electrode that connect to a reservoir.

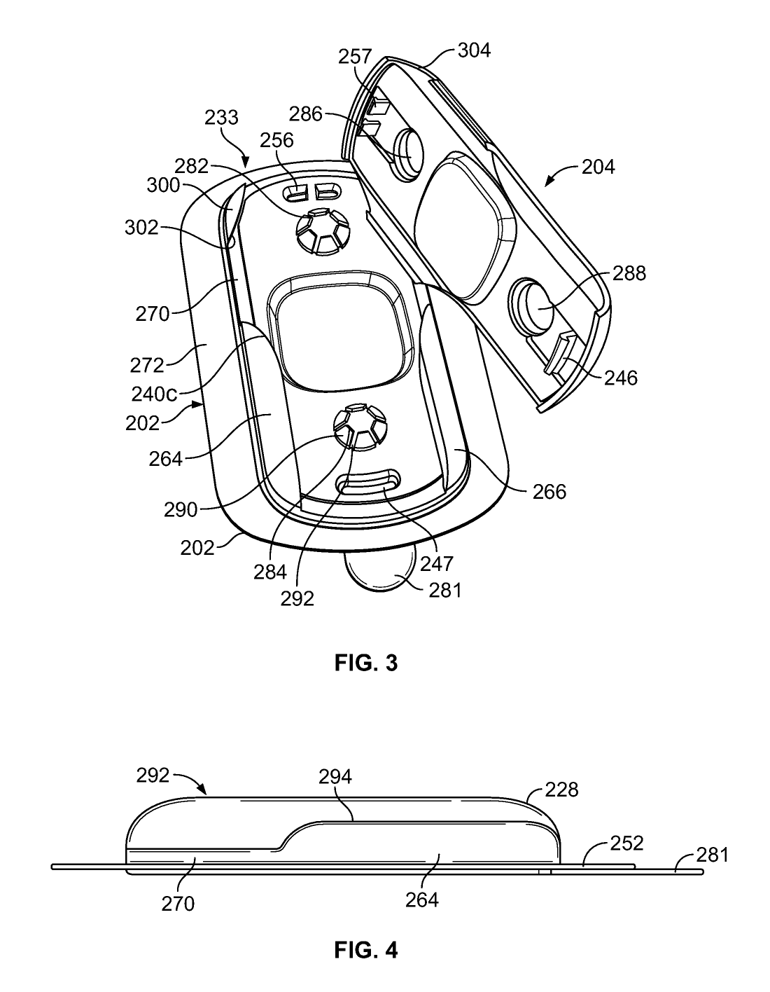

FIG. 3 shows a perspective view showing a device similar to electrotransport device 200 being assembled. In this embodiment, the reservoir module 202 has metallic (which can be a metal, alloy, electroplated material, etc.) electrical connectors 282, 284 for connecting electrically with electrical connectors 286, 288 in the electronic module 204. The metallic electrical connectors 282, 284 shown here have an outward appearance of a generally volcanic mount shape with trapezoidal portions 290 arranged in a ring form with gaps 292 allowing the trapezoidal portions to flex when they are pressed in contact with the electrical connectors 286, 288 of the electronic module 204. The electrical connectors 286, 288 can have a flat surface on which the tip of the electrical connectors 282, 256 can resiliently contact, or can have inserts (e.g., bulb shaped or button shaped) for inserting into the electrical connector 282, 284 of the reservoir module 202 to be grasped by the springing biasing action of the trapezoidal portions 290.

FIG. 3 further shows another feature in an embodiment of the electrotransport device including additional upwardly extending ears 300 from the outer upper portion at the lateral corners about the anterior end 233 of the device (only one ear 300 is shown because the other ear is hidden from view). The upwardly extending ears 300, along with the ridges 264, 266 confine the electronic module 204 about at least the four corners of the device. Since the ears 300 extend from the frame 270 with palms 302 facing at an angle, together with the narrowing portions 240C of the ridges 264 of the outer upper portion 252, the outer upper portion 252 of the reservoir module 202 actually confines the electronic module 204 from movement both longitudinally and laterally, even without taking in consideration the assistance of the couplers 246, 247, 256, 257 and the electrical connectors 282, 284, 286, 288. Thus, once the electronic module 204 and the reservoir module 202 are coupled together, the electronic module 204 is firmly held in place to prevent more than de minimus or nontrivial movement in all directions. The electronic module 204 in FIG. 3 looks substantially similar to those of FIG. 2 and FIG. 5, except for presence of the notches 304 at the corner about the anterior end to accommodate the upwardly extending ears 300.

The upper layer 228 in the electronic module 204 match and fit tightly with the outer upper portion 252 in the reservoir module 202 to result in an inverted-saucer-shaped device 200. FIG. 4 is a schematic side view showing the inverted-saucer-shaped assembled device 200 having a dome 292 rising from the flange 272. The reservoirs are not shown in FIG. 4 because they are hidden from view. FIG. 5 is a top view showing a device similar to the assembled device 200. Although not shown in FIG. 4 and FIG. 5, upwardly extending ears like those of FIG. 3 can also be present. The display 234 in FIG. 5 is a digital display. Alternatively it can be a LED or other light emitting display. The ridges 264 extend up and match fit with the narrowing portion 240A and the narrow portion 242A of the of the upper layer 228 of the upper cover 218 in the electronic module 204 to provide seams 294 that feel and appear like grooves on a surface (preferably continuous surface) to indicate complete and correct assembly under visual and tactile inspection. Although the material on the two sides of the seam can have different thicknesses such that there can be a step appearance, preferably the material on the two sides of the seam butt tightly to be liquid resistant. The anterior portion of the upper layer 228 also match fits with the frame 270 of the outer upper portion 252 to provide seams that are water or liquid resistant. As mentioned, in an alternative, the device shown in FIG. 5 can also include upwardly extending ears 300 like those shown in FIG. 3.

Preferably, the upper layer 228 in the electronic module 204 and the outer upper portion 252 in the reservoir module 202 are both made with the same resilient material so that when the electronic module 204 and the reservoir module 202 are fitted together they form a unit that looks as if it is made of the same material and the dome surface is smooth or continuous (except for the groove like seam) where the ridges 264 meet with the upper layer 228 of the upper cover 218. Thus, the whole device looks like a generally uniform inverted saucer. The inverted-saucer shape is uniform and symmetrical except for the small transparent/translucent window portion 234 for the light or digital display and the small button cover portion 230. Here, the embodiment has an oblong inverted-saucer shape. The button is recessed to help to prevent inadvertent activation of the switch. It is contemplated that other inverted-saucer shapes are possible, e.g., having a planer outline in shapes of circle, polygon, etc.

FIG. 6 shows a side view of the device 200 in the process of being assembled. When the electronic module 204 is to be fitted with the reservoir module 202, one end (e.g., the posterior end 235) of the electronic module 204 can be engaged first. For example, the coupler 246 of the electronic module 204 can loosely engage with the coupler 247 of the reservoir module 202 (e.g., the insert can rest gently on the receptor). By “loosely engaging”, it is meant that the two couplers have not been pressed so firmly together that they become locked and cannot be separated again easily. For couplers with locks (e.g., hooks), the couplers are not matingly engaged yet (if one coupler were fully inserted into the corresponding receptive coupler, then the click or snap fit couplers become locked). Thereafter, the other end (e.g., the anterior end 233) of the device can be engaged. As shown in FIG. 6, for example, with the posterior end 235 loosely engaged, the electronic module 204 and the reservoir module 202 are pressed together at the anterior end 233. The sizing of the channel 267 formed by the ridges 264, 266 and the electronic module 204 is such that the side edges 294 of the upper layer 228 (and optionally the lower layer 226) of the upper cover 218 in the electronic module 204 frictionally contact and slide past the channel wall into the channel 267 (i.e., bound by the walls of the ridges 264, 266) as the electronic module 204 pivots about the posterior end 235 hinging on the couplers 246, 247 to close at the anterior end 233. Thus, the channel walls formed by the ridges 264, 266 provide a visual and mechanical guide for guiding the electronic module 204 and the reservoir module 202 to fit together conveniently.

The narrowing portion 240C of the outer upper portion 252 of the reservoir module 202 provides space to receive the posterior end of the electronic module 204. Thus, a user can first easily place the posterior end of the electronic module 204 into the narrowing portion 240C of the outer upper portion 252 since the narrowing portion 240C is wider than the posterior end of the electronic module 204. Guided by the ridges 264, 266, the user can then easily guide and push the posterior end of the electronic module 204 to slide down the channel 267 from the narrowing portion 240C through the narrow portion 242C toward the posterior end of the reservoir module 202 until the coupler 246 of the electronic module 204 loosely engages the coupler 247 of the reservoir module about the posterior end 235. Then the electronic module 204 and the reservoir module 202 can be pressed together in a pivotal motion to firmly engage the couplers at both the anterior end 233 and the posterior end 235. The couplers can be made with hooks, buttons, barbs, angled ledges, and the like to provide a click-fit or snap-fit to make the engagement permanent when the electronic module 204 and the reservoir modules are firmly pressed together. One kind of insert that can also be used is bar-shaped or has a cross section of a slot U that can be press-fitted to insert into a channel-shaped receptor. In the snap-fit or click-fit, one piece can be inserted into another with a snap or click as a resilient portion of a piece is squeezed initially by a restriction and suddenly released from the squeeze once the portion has passed the restriction. In certain designs, the coupler can be designed so that it is not reengageable. For example, the insert can have a barb or hook that if it is pulled back after insertion into a receptor the barb or hook will rip or distort the receptor so that if reinserted into the receptor the receptor will no longer hold or retain the insert. Alternatively, the barb or hook can be designed to break if pulled so that it cannot reengage with the receptor. Such designs may be used to prevent illicit reuse the device or the reservoir module. If desired, the couplers 246, 247 can be directly and firmly pressed together without first engaging loosely.

In FIG. 5, the embodiment has an electronic module 204 that has a top view that is asymmetric regarding the anterior end 233 and the posterior end 235, although the electronic module is symmetrical regarding its lateral sides. The electronic module 204 has an anterior end that is wider laterally than the posterior end, resulting in a generally key-hole shape. The cutout 268 of the outer upper portion 252 also have a generally key-hole shape to correspond to the shape of the electronic module 204. Thus, a user can easily tell by visual or tactile inspection (or both) which is the anterior end and which is the posterior end on both the electronic module 204 and the reservoir module 202. When the device is needed for application to a patient, the anterior/posterior orientation of the modules can be easily identified by vision or feel for quick assembly. Further, because of their corresponding shapes, there is only one way the electronic module 204 and reservoir module 202 can be fitted together. As used herein, the term “key-hole shaped” refers to a shape similar to key holes for old keys with a big hole on one end having a thinner (usually longer) channel hole branch extending from the big hole.

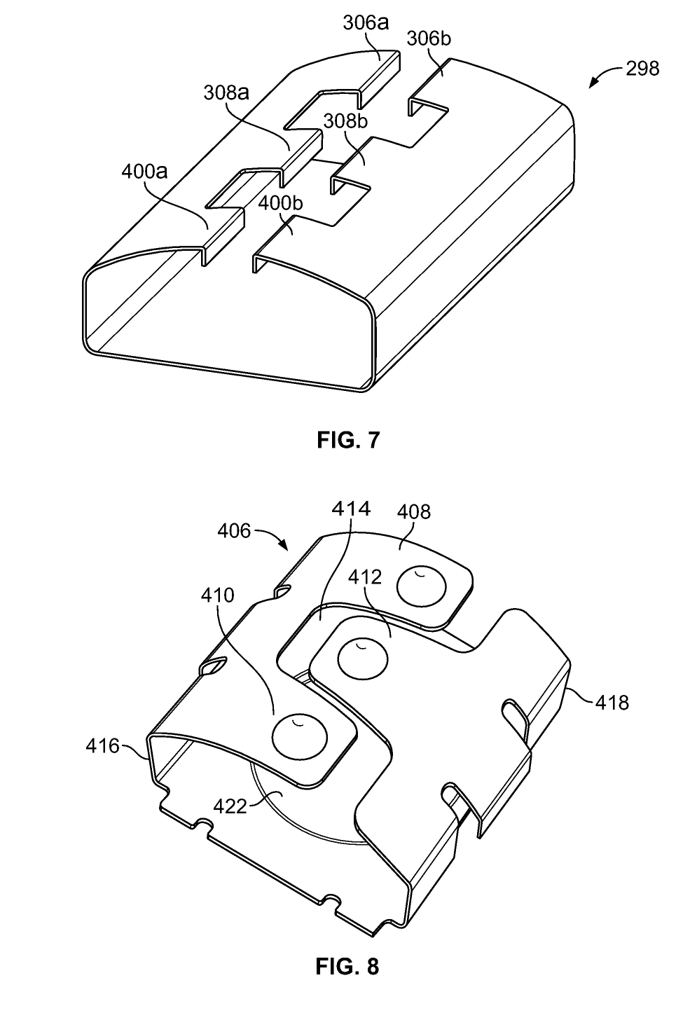

Further embodiments in which other changes are made to the above-described embodiments are possible. For example, FIG. 7 shows another embodiment of an electrical connector that can receive an insert. The electrical connector 298 has a generally female receptor shape for receiving an insert. The electrical connector receptor 298 has fingers 306A, 308A, 400A pointing towards fingers 306B, 308B, 400B and vice versa. An electrical connector insert, which can include a bulb, hook, barb, post, etc., can be inserted between the fingers 306A, 308A, 400A, 306B, 308B, 400B to engage the electrical connector receptor 298 for electrical conduction. Another kind of insert is one that is bar-shaped or has a cross section of a slot U that can be press-fitted to insert into a channel-shaped receptor. Other shapes of inserts and receptors are contemplated so long as they can be coupled together to provide electrical conduction and optionally mechanical retaining engagement when the electronic module 204 and the reservoir module 202 are pressed together. Other than metallic or alloy material, at least some of the electrical connectors can also be made with other conducting material such as carbon, conductive polymers, etc. Furthermore, electroplated or coated materials are also contemplated. Of course, the connector 298 can also offer electrical contact with another electrical connector that simply presses on the face thereof. The resilient (or springy) nature of the connector 298 provides a force to bias the fingers 306A, 308A, 400A, 306B, 308B, 400B towards the other connector to maintain electrical contact. The other connector can also have features that bias toward connector 298, e.g., with a configuration similar to connector 298.

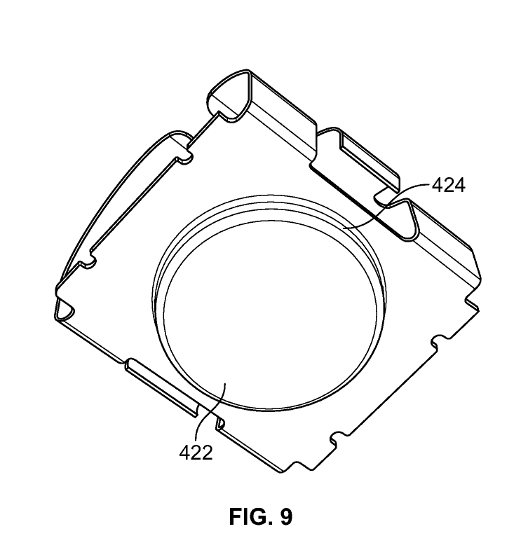

FIG. 8 shows a perspective top view of another embodiment of an electrical connector 406 having bent fingers 408, 410 on one side and finger 412 on an opposite side, each pointing to the other side. FIG. 9 shows a schematic perspective bottom view of the electrical connector of FIG. 8. The fingers 408, 410, 412 are bent at an oblique direction so they have a vector component directing outward (i.e., toward another opposing electrical connector to couple therewith). The fingers 408, 410, 412 are made of a springy material (e.g., metal) that if pressed inward they provide a reacting biasing force outwards. The fingers 408, 410, 412 also are in an interlocking configuration as fingers of two hands interlock such that when pressed, the fingers come together to provide a contact surface with a decreasing gap 414 for better contact with the opposing electrical connector. Supports 416, 418 extend from two opposite sides of a bottom 420 to the fingers 408, 410, 412 to provide room for the fingers to flex. The bottom 420 has a foundation extending further inward to anchor to the inner upper portion 248 of the reservoir module 202. The foundation 422 has a bend 424 from the bottom 420 to provide anchoring to the inner upper portion 248 either by mechanical force and/or by chemical adhesive or bonding.

Although the many of the couplers 246, 247, 256, 257 described above are permanent couplers that once they are firmly pressed together the couplers from the electronic module lock permanently with the corresponding couplers from the reservoir module, it is contemplated that disengageable couplers (e.g., snap button type couplers with a bulb insertable into a receptor hole) can also be used. Devices in which the modules are permanently coupled provide an advantage that the unit is sturdy and the internal electronics and other features are well protected from mechanical disturbance or chemical intrusion and to provide secure electrical and mechanical engagement. Devices in which the modules are separable after use provides the advantage of reusing the electronic module.

The reservoir of the electrotransport delivery devices generally can contain a gel matrix, with the drug solution uniformly dispersed in at least one of the reservoirs. Obviously, other types of reservoirs such as membrane confined reservoirs are possible and contemplated. The application of the present invention is not limited by the type of reservoir used. Gel reservoirs are described, e.g., in U.S. Pat. Nos. 6,039,977 and 6,181,963, which are incorporated by reference herein in their entireties. Suitable polymers for the gel matrix can comprise essentially any synthetic and/or naturally occurring polymeric materials suitable for making gels. A polar nature is preferred when the active agent is polar and/or capable of ionization, so as to enhance agent solubility. Optionally, the gel matrix can be water swellable nonionic material.

Examples of suitable synthetic polymers include, but are not limited to, poly(acrylamide), poly(2-hydroxyethyl acrylate), poly(2-hydroxypropyl acrylate), poly(N-vinyl-2-pyrrolidone), poly(n-methylol acrylamide), poly(diacetone acrylamide), poly(2-hydroxylethyl methacrylate), poly(vinyl alcohol) and poly(allyl alcohol). Hydroxyl functional condensation polymers (i.e., polyesters, polycarbonates, polyurethanes) are also examples of suitable polar synthetic polymers. Polar naturally occurring polymers (or derivatives thereof) suitable for use as the gel matrix are exemplified by cellulose ethers, methyl cellulose ethers, cellulose and hydroxylated cellulose, methyl cellulose and hydroxylated methyl cellulose, gums such as guar, locust, karaya, xanthan, gelatin, and derivatives thereof. Ionic polymers can also be used for the matrix provided that the available counterions are either drug ions or other ions that are oppositely charged relative to the active agent.

Incorporation of the drug solution into the gel matrix in a reservoir can be done in any number of ways, i.e., by imbibing the solution into the reservoir matrix, by admixing the drug solution with the matrix material prior to hydrogel formation, or the like. In additional embodiments, the drug reservoir may optionally contain additional components, such as additives, permeation enhancers, stabilizers, dyes, diluents, plasticizer, tackifying agent, pigments, carriers, inert fillers, antioxidants, excipients, gelling agents, anti-irritants, vasoconstrictors and other materials as are generally known to the transdermal art. Such materials can be included by on skilled in the art.

The drug reservoir can be formed of any material as known in the prior art suitable for making drug reservoirs. The reservoir formulation for transdermally delivering cationic drugs by electrotransport is preferably composed of an aqueous solution of a water-soluble salt, such as HCl or citrate salts of a cationic drug, such as fentanyl or sufentanil. More preferably, the aqueous solution is contained within a hydrophilic polymer matrix such as a hydrogel matrix. The drug salt is preferably present in an amount sufficient to deliver an effective dose by electrotransport over a delivery period of up to about 20 minutes, to achieve a systemic effect. The drug salt typically includes about 0.05 to 20 wt % of the donor reservoir formulation (including the weight of the polymeric matrix) on a fully hydrated basis, and more preferably about 0.1 to 10 wt % of the donor reservoir formulation on a fully hydrated basis. In one embodiment the drug reservoir formulation includes at least 30 wt % water during transdermal delivery of the drug. Delivery of fentanyl and sufentanil has been described in U.S. Pat. No. 6,171,294, which is incorporated by reference herein. The parameter such as concentration, rate, current, etc. as described in U.S. Pat. No. 6,171,294 can be similarly employed here, since the electronics and reservoirs of the present invention can be made to be substantially similar to those in U.S. Pat. No. 6,171,294.

The drug reservoir containing hydrogel can suitably be made of any number of materials but preferably is composed of a hydrophilic polymeric material, preferably one that is polar in nature so as to enhance the drug stability. Suitable polar polymers for the hydrogel matrix include a variety of synthetic and naturally occurring polymeric materials. A preferred hydrogel formulation contains a suitable hydrophilic polymer, a buffer, a humectant, a thickener, water and a water soluble drug salt (e.g. HCl salt of an cationic drug). A preferred hydrophilic polymer matrix is polyvinyl alcohol such as a washed and fully hydrolyzed polyvinyl alcohol (PVOH), e.g. MOWIOL 66-100 commercially available from Hoechst Aktiengesellschaft. A suitable buffer is an ion exchange resin which is a copolymer of methacrylic acid and divinylbenzene in both an acid and salt form. One example of such a buffer is a mixture of POLACRILIN (the copolymer of methacrylic acid and divinyl benzene available from Rohm & Haas, Philadelphia, Pa.) and the potassium salt thereof. A mixture of the acid and potassium salt forms of POLACRLIN functions as a polymeric buffer to adjust the pH of the hydrogel to about pH 6. Use of a humectant in the hydrogel formulation is beneficial to inhibit the loss of moisture from the hydrogel. An example of a suitable humectant is guar gum. Thickeners are also beneficial in a hydrogel formulation. For example, a polyvinyl alcohol thickener such as hydroxypropyl methylcellulose (e.g. METHOCEL K100 MP available from Dow Chemical, Midland, Mich.) aids in modifying the rheology of a hot polymer solution as it is dispensed into a mold or cavity. The hydroxypropyl methylcellulose increases in viscosity on cooling and significantly reduces the propensity of a cooled polymer solution to overfill the mold or cavity.

Polyvinyl alcohol hydrogels can be prepared, for example, as described in U.S. Pat. No. 6,039,977. The weight percentage of the polyvinyl alcohol used to prepare gel matrices for the reservoirs of the electrotransport delivery devices, in certain embodiments can be about 10% to about 30%, preferably about 15% to about 25%, and more preferably about 19%. Preferably, for ease of processing and application, the gel matrix has a viscosity of from about 1,000 to about 200,000 poise, preferably from about 5,000 to about 50,000 poise. In certain preferred embodiments, the drug-containing hydrogel formulation includes about 10 to 15 wt % polyvinyl alcohol, 0.1 to 0.4 wt % resin buffer, and about 1 to 30 wt %, preferably 1 to 2 wt % drug. The remainder is water and ingredients such as humectants, thickeners, etc. The polyvinyl alcohol (PVOH)-based hydrogel formulation is prepared by mixing all materials, including the drug, in a single vessel at elevated temperatures of about 90 degree C. to 95 degree C. for at least about 0.5 hour. The hot mix is then poured into foam molds and stored at freezing temperature of about −35 degree C. overnight to cross-link the PVOH. Upon warming to ambient temperature, a tough elastomeric gel is obtained suitable for ionic drug electrotransport.

A variety of drugs can be delivered by electrotransport devices. In certain embodiments, the drug is a narcotic analgesic agent and is preferably selected from the group consisting of fentanyl and related molecules such as remifentanil, sufentanil, alfentanil, lofentanil, carfentanil, trefentanil as well as simple fentanyl derivatives such as alpha-methyl fentanyl, 3-methyl fentanyl and 4-methyl fentanyl, and other compounds presenting narcotic analgesic activity such as alphaprodine, anileridine, benzylmorphine, beta-promedol, bezitramide, buprenorphine, butorphanol, clonitazene, codeine, desomorphine, dextromoramide, dezocine, diampromide, dihydrocodeine, dihydrocodeinone enol acetate, dihydromorphine, dimenoxadol, dimeheptanol, dimethylthiambutene, dioxaphetyl butyrate, dipipanone, eptazocine, ethylmethylthiambutene, ethylmorphine, etonitazene, etorphine, hydrocodone, hydromorphone, hydroxypethidine, isomethadone, ketobemidone, levorphanol, meperidine, meptazinol, metazocine, methadone, methadyl acetate, metopon, morphine, heroin, myrophine, nalbuphine, nicomorphine, norlevorphanol, normorphine, norpipanone, oxycodone, oxymorphone, pentazocine, phenadoxone, phenazocine, phenoperidine, piminodine, piritramide, proheptazine, promedol, properidine, propiram, propoxyphene, and tilidine.

Some ionic drugs are polypeptides, proteins, hormones, or derivatives, analogs, mimics thereof. For example, insulin or mimics are ionic drugs that can be driven by electrical force in electrotransport.

For more effective delivery by electrotransport salts of certain pharmaceutical analgesic agents are preferably included in the drug reservoir. Suitable salts of cationic drugs, such as narcotic analgesic agents, include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, β-hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate. The more preferred salt is chloride.

A counterion is present in the drug reservoir in amounts necessary to neutralize the positive charge present on the cationic drug, e.g. narcotic analgesic agent, at the pH of the formulation. Excess of counterion (as the free acid or as a salt) can be added to the reservoir in order to control pH and to provide adequate buffering capacity. In one embodiment of the invention, the drug reservoir includes at least one buffer for controlling the pH in the drug reservoir. Suitable buffering systems are known in the art.

Obviously, the present invention is also applicable where the drug is an anionic drug. In this case, the drug is held in the cathodic reservoir (the negative pole) and the anoidic reservoir would hold the counterion. A number of drugs are anionic, such as cromolyn (antiasthmatic), indomethacin (anti-inflammatory), ketoprofen (anti-inflammatory) and ketorolac tromethamine (NSAID and analgesic activity), and certain biologics such as certain protein or polypeptides.

### Method of Making

A device according to the present invention can be made by forming the layers separately and assembling the layers into the electronic module and the reservoir module. The polymeric layers can be made by molding. Some of the layers can be applied together and secured. Some of the layers can be comolded, for example, by molding a second layer onto a first layer. For example, the upper layer and lower layer of the upper cover (or top cover) can be comolded together. Some of the layers can be affixed together by adhesive bonding or mechanical anchoring. Such chemical adhesive bonding methods and mechanical anchoring methods are known in the art. As described before, once the electronic module and the reservoir module are formed, they can be packaged separately. Before use, the two modules can be removed from their respective packages and assembled to form the device for electrotransport. The device can then be applied to the body surface by adhesion.

The above-described exemplary embodiments are intended to be illustrative in all respects, rather than restrictive, of the present invention. Thus the present invention is capable of many variations in detailed implementation that can be derived from the description contained herein by a person skilled in the art, e.g., by permutation or combination of various features. Although iontophoretic devices are described in detail as illustration for showing how an electronic module and an agent module are coupled and work together, a person skilled in the art will know that electronic module and agent module in other electrotransport devices can be similarly coupled and work together. All such variations and modifications are considered to be within the scope of the present invention. The entire disclosure of each patent, patent application, and publication cited or described in this document is hereby incorporated herein by reference.

We claim:

1. An electrotransport device for delivering a therapeutic agent through a body surface of a patient, the device comprising:
agent module having a first end, a second end, and including a compartment containing the therapeutic agent for electrotransporting through the body surface, the agent module further including a coupler and including a cutout forming a channel having a narrow channel portion and a less narrow channel portion with channel walls;
electronic module having a first end and a second end corresponding to the first end and second end of the agent module, and including a coupler for coupling with corresponding coupler of the agent module, the electronic module having a body with a narrow portion and a less narrow portion corresponding to the narrow portion and the less narrow portion of the channel in the agent module, the narrow portion of the electronic module terminating about its first end, the electronic module including circuitry for electrically driving the therapeutic agent for electrotransport.

2. The device of claim 1, wherein the first end of the electronic module can be received and guided by the channel walls at the less narrow channel portion into the narrow channel portion to align with the agent module, the coupler of the electrical module having at least one of a projection and a receptor for coupling with at least one of a projection and a receptor in corresponding coupler of the agent module, wherein the compartment is a reservoir.

3. The device of claim 2, wherein the agent module and electronic module can engage at one end before engaging another end so that the modules at the another end can move closer together in a pivoting motion.

4. The device of claim 2, wherein the agent module and electronic module can engage about one end before engaging another end so that the modules at the another end can move closer together by a pivoting motion such that at least a portion the electronic module frictionally slide on the channel wall as it is guided into the channel in the pivotal motion.

5. The device of claim 2, wherein the agent module and electronic module can engage about one end by engaging a projection of one of said agent module and said electronic module with a receptor of the other module before engaging about another end.

6. The device of claim 2, wherein the agent module has at least two upwardly extending ridges about the first end and two upwardly extending projections about the second end for constraining the electronic module, the two upwardly extending ridges about the first end having lengths different from lengths of the two upwardly extending projections about the second end and wherein the electronic module has a wide portion between the first end and second end and not constrained by the two upwardly extending ridges and the two upwardly extending projections of the agent module.

7. The device of claim 2, wherein the agent module has a gradually narrowing channel portion and the electronic module has a gradually narrowing portion matching the gradually narrowing channel portion of the agent module, the agent module having two upwardly extending ridges from about the first end or second end for constraining the electronic module, the two upwardly extending ridges extending longitudinally to at least half of the agent module to define at least a portion of the channel.

8. The device of claim 1, wherein the electronic module includes a printed circuit board (PCB) having circuitry for controlling the function of the device, an upper cover and a lower cover protecting the PCB in the middle.

9. The device of claim 1, wherein the electronic module includes a printed circuit board (PCB) having circuitry for controlling the function of the device, an upper cover and a lower cover protecting the PCB in the middle and whereas electrical connectors for connecting with the agent module are one of being covered by a conductive pads of the lower cover or not covered by the lower cover.

10. The device of claim 1, wherein the electronic module includes a printed circuit board (PCB) having circuitry for controlling the function of the device, an upper cover and a lower cover protecting the PCB in the middle, the upper cover having a polymeric material less rigid than the material of the lower cover such that the upper cover can match the agent module to be liquid resistant.

11. The device of claim 1, wherein the agent module includes a rigid member supporting electrical connectors for connecting with electrical connectors from the electronic module and includes a less rigid portion supporting the rigid member, the less rigid portion allowing the device to be placed on skin.

12. The device of claim 1, wherein the agent module includes a rigid member supporting electrical connectors for connecting with electrical connectors from the electronic module and includes a less rigid portion supporting the rigid member, the less rigid portion including the channel wall for receiving at least a portion of the electronic module and match with an upper cover of the electronic module to be liquid resistant.

13. The device of claim 1, wherein the agent module includes a rigid member supporting electrical connectors for connecting with electrical connectors from the electronic module and includes a less rigid portion supporting the rigid member, the less rigid portion including the channel wall for receiving the electronic module, the channel wall having a widening portion, the electronic module having a printed circuit board (PCB) having circuitry for controlling the function of the device, an upper cover and a lower cover protecting the PCB in the middle, the upper cover having a polymeric material less rigid than the material of the lower cover and having a narrowing portion such that the upper cover can match the agent module to be liquid resistant.

14. The device of claim 2, wherein the electronic module has a molded cover having a top softer opaque portion and a less soft light transmitting portion under the top softer opaque portion.

15. The device of claim 2, wherein the electronic module and the agent module when assembled form an inverted saucer shaped assembly having flat annular rim surrounding a dome, the flat annular rim being flexible to conform to human skin surface, the dome having a surface being formed partly from the electronic module and partly from the agent module and the inverted saucer shaped assembly has a top surface essentially all of which are of the same material as the flat annular rim.

16. A method of making an electrotransport device for delivering a therapeutic agent through a body surface of a patient, comprising coupling an agent module to an electronic module, wherein the agent module has a first end, a second end, and including a compartment which contains the therapeutic agent for delivery through the body surface, the agent module including a coupler, the agent module further including a cutout forming a channel having a narrow channel portion and a less narrow channel portion with channel walls; the electronic module having a first end and a second end corresponding to the first end and second end of the agent module, and a coupler for coupling with corresponding coupler of the agent module, the electronic module including circuitry for electrically driving the therapeutic agent for electrotransport, the electronic module having a body with a narrow portion and a less narrow portion corresponding to the narrow channel portion and less narrow channel portion of the agent module, the narrow portion of the electronic module terminating about the first end; the method further comprising guiding the less narrow portion and the narrow portion of the electronic module into the less narrow channel portion and narrow channel portion of the agent module.

17. The method of claim 16, wherein the first end of the electronic module can be received and guided by the channel walls at the less narrow channel portion into the narrow channel portion to align with the agent module, the electrical module coupler having at least one of a projection and a receptor for coupling with corresponding at least one of a projection and a receptor in the coupler of the agent module.

18. The method of claim 17, wherein the agent module and electronic module can engage about one end before engaging another end so that the modules about the another end can move closer together by a pivoting motion.

19. The method of claim 17, wherein the agent module and electronic module can engage about one end before engaging another end so that the modules about the another end can move closer together by a pivoting motion such that at least a portion of the electronic module slides on the channel wall as it is guided into the channel in the pivotal motion.

20. The method of claim 17, wherein the agent module has a gradually narrowing channel portion and the electronic module has a gradually narrowing portion matching the gradually narrowing channel portion, the agent module having two upwardly extending ridges about the first end or second end for constraining the electronic module, the two upwardly extending ridges run longutudinally to at least half of the agent module.

21. The method of claim 16, wherein the agent module includes a rigid upper member with electrical connectors for connecting with electrical connectors from the electronic module and a less rigid portion supporting the rigid upper member, the less rigid portion allowing the device to be placed on skin and matching contour with the electronic module.

22. The method of claim 17, further comprising removing the electrical module from a package and removing the agent module from another package before coupling the two modules.

23. A method of making an electrotransport device for delivering a therapeutic agent through a body surface of a patient, comprising:
removing an agent module from a package, the agent module has a first end, a second end, and including a compartment which contains the therapeutic agent for electrotransporting through the body surface, the agent module including a coupler, the agent module further including a cutout forming a channel having a narrow channel portion and a less narrow channel portion with channel walls;
removing an electronic module from another package, the electronic module having a first end and a second end corresponding to the first end and second end of the agent module, and a coupler for coupling with the corresponding coupler of the agent module, the electronic module having a body with a narrow portion and a less narrow portion corresponding to the narrow channel portion and less narrow channel portion of the agent module, the electronic module including circuitry for electrically driving the therapeutic agent for electrotransport; and
coupling the agent module to the electronic module.
